(19) United States
(12) Patent Application Publication
(10) Pub. No.: US 2006/0074254 A1
(43) Pub. Date: Apr. 6, 2006

(54) Process for extracting taxanes

(76) Inventors: Zisheng Zhang, Ottawa (CA); Yuheng Wang, Edmonton (CA)

Correspondence Address: KIRBY EADES GALE BAKER, BOX 3432, STATION D, OTTAWA, ON, K1P 6N9, CA

(21) Appl. No.: 11/239,011

(22) Filed: Sep. 30, 2005

Related U.S. Application Data
(60) Provisional application No. 60/614,429, filed on Sep. 30, 2004.

(51) Int. Cl. C07D 305/14

(52) U.S. Cl. 549/510

(57) ABSTRACT

A method of extracting taxane products from biomass, which involves feeding the biomass into an pressurized liquid extraction unit and contacting the biomass with a halogenated C₁ to C₂ alkane solvent at a temperature of 100° C. or less and at sufficient pressure to keep the solvent in liquid form, to extract a stream of taxanes and solvent. The stream of taxanes and solvent are then cooled arid the solvent is stripped from the taxanes. The taxanes are finally passed through either a normal phase liquid chromatograph or a reverse phase liquid chromatograph.

### CROSS REFERENCE TO RELATED APPLICATION

This application claims the priority right of prior U.S. patent application Ser. No. 60/614,429 filed on Sep. 30, 2004 by applicants herein.

### FIELD OF THE INVENTION

The present invention relates to a method of extracting taxane products, and more specifically to methods of extracting taxane products from biomass materials.

### BACKGROUND OF THE INVENTION

In recent years taxanes, particularly in the form of paclitaxel, have been found to be highly effective agents in cancer treatment. In particular, paclitaxel has been successfully used in treating breast, ovarian and n-on-small cell lung cancer. Taxanes come from the bark of the yew tree (e.g. Taxus canadensis) and are naturally found in very low concentrations of between 100 and 300 ppm in the tree material. The use of taxanes as an effective ingredient in the treatment of cancer has lead to a great demand for recovering these products from the yew tree with as high yield as possible.

Many methods for increasing the production of taxanes have been explored in the past number of years. These methods include attempts to maximize the growth of yew trees by farming, synthesising taxanes through chemistry techniques, exploring biotechnological techniques such as fermentation and cell cultures, and using extraction and bioseparation technologies.

Traditionally, natural product extraction from the biomass of the yew tree has been the first step in the production of taxane products. However, extraction is often also the limiting step in mass production. This is because of the very low concentrations of taxanes in the dry needle and twig of the yew tree. Typically, large amounts of organic solvents are required for such extractions. Several state-of-the-art technologies, such as sonication and microwave-assisted extraction, have also been tested, but none have proven efficient for commercial production. Very often, extraction and separation steps make up 80% of the total manufacturing cost of plant-based medicines.

U.S. Pat. No. 6,469,186 by Kasitu et al., teaches a process of paclitaxel extraction using lower alcohols or mixtures thereof, as solvents. The process however, requires a number of steps, including separate extraction and concentration steps, which can lead to product degradation.

In U.S. Pat. No. 5,843,311, accelerated solvent extraction (ASE) is conducted at elevated temperatures and high pressure above 100 psig. The high pressure is required to enable the solvent to dissolve air inside pores c)f the biomass material, so that the solvent can contact the taxane products, while keeping the organic solvents in liquid form at the elevated temperature. The very high operating pressure adds a large cost to taxane production, making the ASE method unfeasible for large-scale commercial production.

Supercritical fluid extraction (SFE), as described in U.S. Pat. No. 6,503,396 results in less environmental impact than ASE. However, the selectivity of this method is no better than that of ordinary solvent extraction for taxane isolation from biomass. With typical operating pressures of as high as 600 atm, the SFE system is also costly to build and operate, making it less suitable for extraction of low concentration products, such as taxane.

Almost all the existing processes for taxane mass production begin with ordinary solvent extraction (OSE). Organic solvents commonly used include methanol, ethanol or mixtures of methylene chloride and methanol These solvents have very low selectivity, and tend to extract large quantities of lipids and by-products along wit:h the taxane. The weight of methanol extract can be as much as 53% of that of dry needles of Taxus canadensis. This is a good indication that lipids and by-products have been extracted as well, since the fraction of taxanes in the yew tree is only 100 ppm to 300 ppm. The use of mixed solvents also often causes solvent recovery problems.

Very often, to concentrate heat sensitive products such as paclitaxel from the resultant extracts, a vacuum must be applied. This can lead to losses of both solvents and products if the products are dissolved during the operation.

Because of the low selectivity of ordinary solvent extraction, several unit operations must be applied to remove impurities from the extracts before feeding the product to a normal or reverse phase liquid chromatography column. These steps commonly include a separate lipid extraction step before solvent extraction. Since each process step achieves less than 100% recovery of the products, the overall recovery rates of the products: decreases with each additional process step.

As discussed above, numerous primary steps are required before the liquid chromatography step, due to the low selectivity of the OSE step. The overall recovery of taxanes in processes based on OSE is estimated to be very low. Additionally, the major product, paclitaxel, a heat-sensitive and readily degraded during processing, so that additional process steps often act to degrade the desired product.

The operating and capital costs of SFE-based processes are typically higher than many existing extraction techniques and can only be acceptable for commercial production if better selectivity can be achieved. To achieve better selectivity, a large amount of co-solvent such as ethanol, is required, which leads to additional steps of separating the solvent mixture components for regeneration.

It is therefore greatly desirable to develop a process for taxane product extraction that results in low operating costs and high product yield. It is also desirable to find ways of integrating individual unit operations and extraction steps.

### SUMMARY OF THE INVENTION

The present invention provides an integrated process for extracting taxanes from plant materials. The process comprises comminuting taxanes-containing biomass and feeding the biomass into a dynamic pressurized liquid extraction unit and contacting the biomass with a halogenated C₁ or C₂ alkane at a temperature of 100° C. or less and at sufficient pressure to keep the solvent in liquid form, to extract a stream of taxanes and solvent. The stream of taxanes and solvent is then cooled and the solvent is stripped from the taxanes. Finally, liquid chromatography is conducting on the taxanes to purify the taxanes.

The present invention also provides a way of using dynamic pressurized liquid extraction in a process for extracting taxanes from plant biomass.

The present invention further provides a process of selecting at least one solvent for extracting a product from plant biomass containing a plurality of compounds, by assessing relative hydrophobicity of the compounds, arranging the compounds on a scale of most hydrophobic to least hydrophobic and matching the hydrophobicity of the product to the hydrophobicity of the at least one solvent.

### BRIEF DESCRIPTION OF THE DRAWINGS

The present invention will be described in conjunction with the following figures, wherein:

FIG. 1 is a schematic diagram of a prior art process for taxane extraction;

FIGS. 2a to 2e are graphical representations of the hydrophobic and hydrophilic constituents of the plant biomass;

FIG. 3 is a schematic diagram of one embodiment of the process of the present invention;

FIG. 4 is a schematic diagram of another embodiment of the process of the present invention;

FIG. 5 is a graph showing the effect of solvent flow rate on taxane extraction; and

FIG. 6 is a schematic diagram of a test set-up for testing embodiments of the process of the present invention.

### DETAILED DESCRIPTION OF THE PREFERED EMBODIMENTS

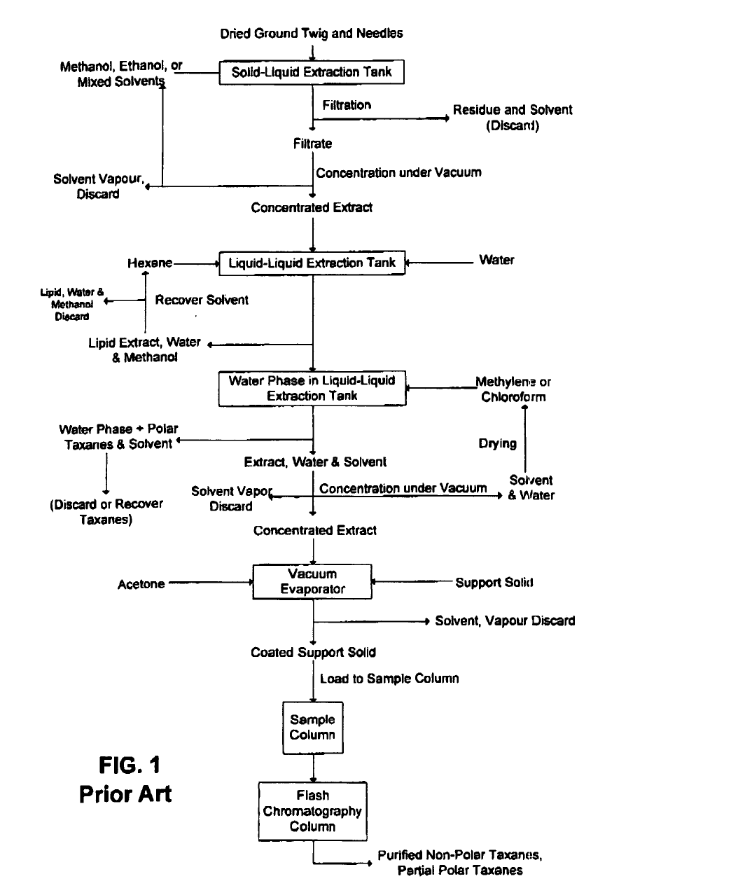

For purposes of comparison, FIG. 1 shows a typical process that is based on ordinary solvent extraction (OSE). The figure is believed to be self-explanatory. The process of FIG. 1 has up to 7 unit operations before the liquid chromatography step and a large amount of polar taxanes, such as 10-DAB III, are lost in wastewater during the solvent-solvent extraction step.

The process of the present invention is based on the inventors' observations that certain solvents have it greater selectivity towards taxane products, resulting in fewer by-products being picked up in the initial extraction step. This in turn means that less processing is required after extraction to purify and concentrate the desired taxanes. The result is a more integrated extraction process, having fewer unit operations than traditionally seen.

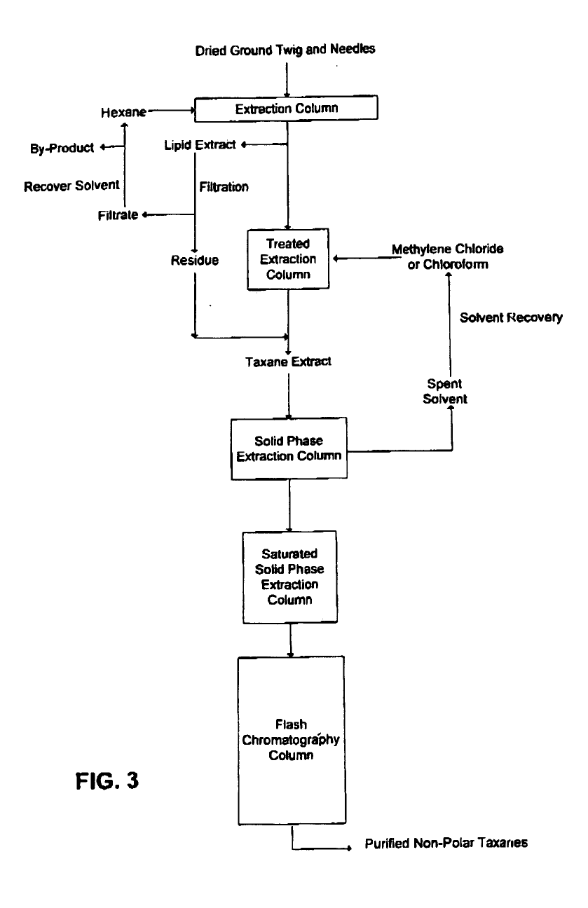

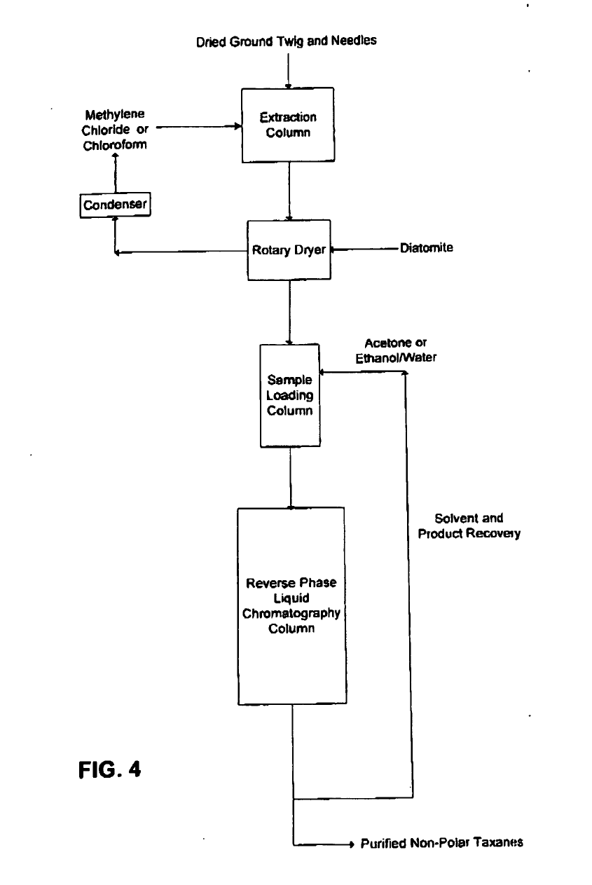

FIGS. 3 and 4 generally illustrate embodiments of the present invention for the isolation and purification of taxanes, involving the following steps:
a) The starting biomass is dried by means known in the art, such as air drying, and then reduced to small particles by grinding, pulverizing or crushing. The starting biomass can include any taxanes-containing plants, including Taxus brevifolia, Taxus canadensis, Taxus baccata, et al.
b) Dynamic Pressurized Liquid Extraction (DPLE) is conducted on the dried biomass under low pressure and low temperature. The temperature is generally 100° C. or less and the pressure need only be just high enough to keep the solvent in liquid form. The solvent for the extraction step is a solvent having high selectivity to the taxanes products, such as a halogenated C₁ or C₂ alkane.
c) The resulting taxane product/solvent stream is cooled to lower the stream temperature.
d) The solvent can be removed from the taxane product by any suitable method known in the art, including solid phase extraction (SPE), evaporation, or adsorption and washing.
e) Once taxane products have been isolated, the taxanes are passed through either a normal phase or reverse phase liquid chromatography column.

In preparing the biomass material, the preferred parts are twig and needles, which are renewable sources. These are preferably ground to a particle size finer than 100 mesh.

The preferred temperature for the dynamic pressurized liquid extraction step is in the range of 50-100° C., and more preferably from 80-100 ° C. If the temperature is too low, not all of the taxanes are effectively extracted. Conversely, if the temperature is too high, there is an increased chance of undesired impurities and lipids being extracted with the taxanes. The pressures and temperatures are lower than those generally used in ASE processes, which are typically conducted at about 1500 psig and more than 100° C.

The halogenated C₁ to C₂ alkane is preferably halogenated by chlorine. Preferred solvents in the DPLE step are methylene chloride and chloroform, which show high selectivity towards the taxane products.

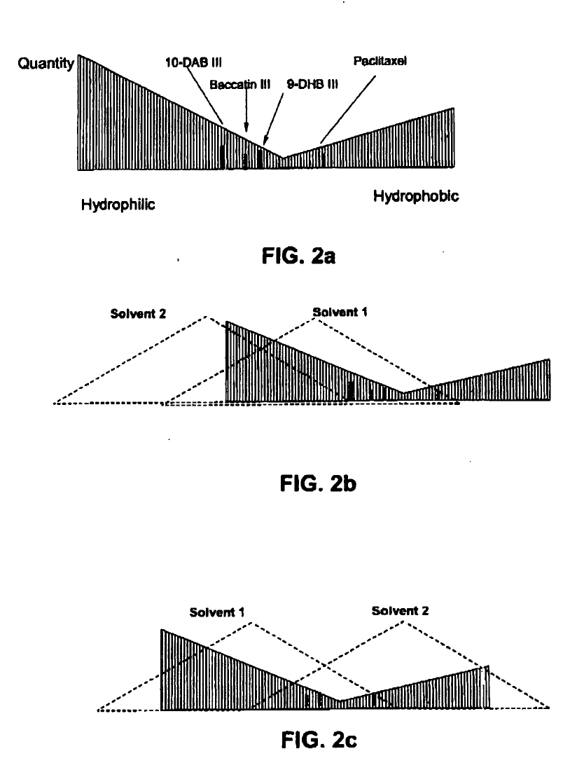

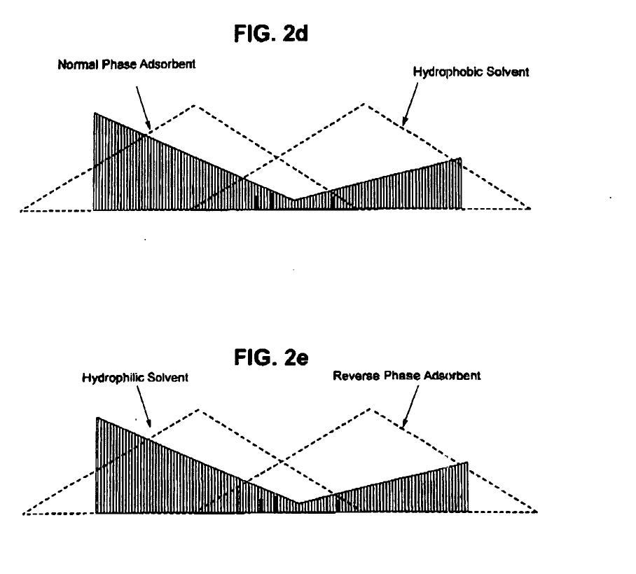

It is important to choose solvents that have a high selectivity to the desired products in the biomass. Based on the common knowledge that solvents dissolve products that have similar properties to the solvent, the present inventors have examined properties of compounds contained in the biomass, in particular the relative hydrophobicities of the compounds. FIG. 2a shows, for example, the compounds contained in yew tree biomass on a scale of most hydrophilic to most hydrophobia. In this case, the desired taxane products fall generally in the middle of this scale, paclitaxel being slightly more hydrophobic and the other taxanes products being slightly more hydrophilic. Relative hydrophobicities of the compounds can be determined through any known means and can be assessed by for example, chromatography. Solvents are then selected by matching the hydrophobicity of the product to be extracted with the hydrophobicity of one or more solvents.

A first optional method for selecting solvents illustrated in FIG. 2b, is to choose a first solvent that extracts all of the desired taxanes, together with all of the impurities from one end of the scale. Next, a second solvent is chosen with an affinity to these impurities, thereby leaving behind the taxanes. A second optional method in choosing solvents, illustrated in FIG. 2c, is to choose a first solvent to extract the taxanes plus all impurities from one end of the scale and then to extract with a second solvent that has an affinity to the taxanes and everything on the other end of the scale, thereby separating the taxanes from the impurities.

In a third optional method for selecting solvents, shown in FIG. 2d, taxanes are extracted by hydrophobic solvents and then selectively adsorbed on the surface of a normal phase adsorbent, such as for example, silica gel, by normal phase solid phase extraction (NP-SPE) while the hydrophobic impurities are left behind. NP-SPE can be followed by normal phase preparative chromatography to yield taxanes with high purity.

In a fourth optional method for selecting solvents, shown in FIG. 2e, taxanes are extracted by hydrophilic solvents and then selectively adsorbed on the surface of a reverse phase adsorbent, such as for example, resin or activated charcoal, by reverse phase solid phase extraction (RP-SPE) while the hydrophilic impurities are left behind. RP-SPE can also be followed by reverse phase preparative chromatography to obtain high purity taxanes.

The above methods are not restricted to extraction of taxanes from yew tree biomass, and can be applied in selecting solvents for extracting any products from any type of biomass.

The extraction process can be conducted in a column or tank and the preferred form is in a column. Although pressurized liquid extraction can be conducted as a batch process, a dynamic process is preferred.

Dynamic pressurized liquid extraction (DPLE) is a process of continuously feeding solvent into the extraction column, while continuously drawing the extract stream out from the column. In DPLE, solvents are continuously passed thought the matrices of the biomass. This continuous stream of solvent dissolves any water or air that often fills the matrices and blocks the solvents from reaching the solutes. Therefore, compared to PLE, high pressure is not required to force the solvents into the matrices occupied by water or air, to access the solutes. Only enough pressure is required to prevent solvents from boiling at operating temperatures.

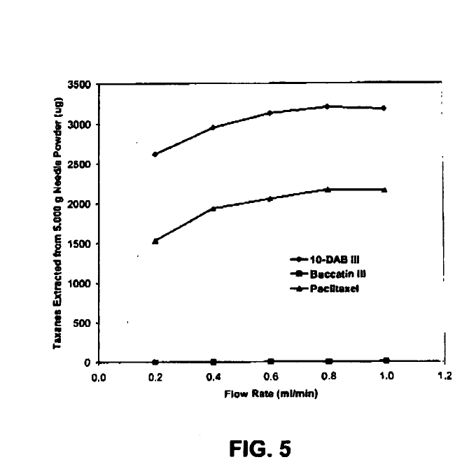

As well, the extraction efficiency of DPLE exceeds that of PLE because fresh solvent is continuously introduced into the extraction column. The mass transfer rate inside the column is accelerated by increased concentration difference between the fresh solvent and the solute in the biomass. The increase in taxanes extraction with increased flow rates is illustrated in FIG. 5 Furthermore, DPLE allows for a continuous stream of taxanes to be produced and eliminates the common delays of charging and unloading the column that occur in traditional batch productions.

Optionally, a lipid extraction step can be performed before DPLE, to remove oils and dyes from the ground yew particles. These oils and dyes often contain substances such as chlorophyll and vitamin E, which can be used to produce natural chlorophyll and vitamin E products. By separating these substances before DPLE is conducted, they can be sold as valuable by-products. Lipid extraction is performed with a solvent such as petroleum ether or hexane and the preferred temperature for lipid extraction is in the range of 70-100° C. A more preferred temperature range is 90-100° C. The optional step of lipid extraction is shown in FIG. 3.

An inert gas purge is optionally conducted before lipid extraction (not shown), to remove oxygen from inside the lipid extraction column. A gas purge can optionally also be conducted after each of the lipid extraction and dynamic pressurized liquid extraction steps to remove any traces of solvent used in each of these steps, thereby avoiding solvent carry-over to subsequent steps. A preferred gas for use in purging is nitrogen. The solvent-containing inert gas can then be recovered by adsorption, preferably onto an active carbon fibre matrix, which is well known in the art.

The dynamic pressurized liquid extraction step produces an extract stream comprising taxane products, solvent and trace impurities. There are two preferred options after dynamic pressurized liquid extraction for removing solvent from the taxane product. The first option is illustrated in FIG. 3 and the second option is illustrated in FIG. 4.

In the first option, solid phase extraction is conducted to separate the spent solvent from the taxane products. The adsorbent material in the packed solid phase extraction column can be any normal phase material, and is preferably silica gel or Al₂O₃. In this first option, the extract passes through the packed column, and the solvent is separated from the taxanes and recovered for reuse. The solutes (taxanes and trace impurities) are adsorbed on the silica gel or Al₂O₃ and are purified by gradient elution, optionally using methylene chloride plus other polar solvents known in the art. Alternatively, the solutes can be directly loaded to a normal phase chromatography column.

In an optional embodiment, the solid phase extraction column is further treated to remove any impurities before the taxanes are loaded into chromatography column.

After adsorption, the solid phase extraction column is optionally purged by an inert gas such as nitrogen to remove any halogenated solvent from the adsorption media, which can contaminate the downstream normal phase chromatography process.

In the second option, as illustrated by FIG. 4, the extract stream is introduced into a continuous rotary dryer, along with a porous solid support material such as diatomite (for example Celite 545™, usually used as filtration aid). The mass flow ratio of solids in the extract to diatomite should be kept between 1:10 and 1:3. The solvent used in ASE is typically low-boiling and is therefore easily evaporated in the dryer. In the case of methylene chloride, the boiling point is just 44° C. at 1 atm. The solvent is then condensed and reused in the extraction process. The solute, comprising the taxane products and trace impurities, are left on the surface of numerous pores in the support material.

The support material with the taxanes and trace impurities are loaded into a sample column. The impurities are mainly comprised of lipids, such as chlorophyll. The column is eluted with an organic solvent/water mixture such as, for example ethanol/water, to remove all taxanes from she coated material. Most of the impurities are not eluted out and remain on the surface of the support material.

The eluant, comprising eluted taxanes in the ethanol/water mixture, is forced through a reverse phase chromatography column and the taxanes are adsorbed say reverse phase chromatography. Taxanes are generally absorbed on the top of the packing of the column. Then gradient elution is conducted to obtain purified taxanes, such as 10-DAB 3, paclitaxel, and 9-DIM 3.

The preferred reverse phase packing comprises macro pyre resins. The preferred products recovery method after reverse phase chromatography is by membrane separation.

The reverse phase chromatography based process presented in FIG. 4 is generally most preferred. This is because almost all of the solvent is kept in the extraction loop, leaving almost no solvent residue in the biomass after extraction. As well, the ethanol/water in the eluant of reverse phase chromatography is considered a suitably mild substance that will not cause taxane degradation.

Finally, the lipids, which can be harmful to reverse phase absorbents, are left out of the chromatography column, thus also lowering the total mass loaded to the chromatography column. Liquid-liquid extraction is generally quite acceptable in the industry as a means to remove lipids to avoid destroying the reverse phase absorbents.

The steps of concentration and extraction in the present invention are integrated and conducted at the same time and there is no need for a vacuum system. There are only two solvents required in the whole process, and no mixing of solvents is required. Solvents recovery is thus much simpler than that of solvent mixtures.

The extract stream, containing taxanes, solvent and trace impurities, is only about 8% (w/w) based on the weight of dry twig and needles, compared to 53% (w/w) in the prior art technologies, showing the high selectivity in the extraction step of the present invention. The high selectivity means that no further treatment is necessary before the step of liquid chromatography. As a result, the whole process comprises only 2 or 3 unit operations compared to that of up to 7 unit operations in prior art technologies.

The steps of the present invention are further illustrated by the following examples.

### EXAMPLE I

Fresh twig and needles of Taxus canadensie were picked at Hartland and Rexton, New Brunswick, Canada in May, 2003. After drying for 7 days in darkness at ambient temperature and humidity, the needles were stripped manually from stems and ground to a powder with particles finer than 20 mesh. The ground needle powder was refrigerated at a temperature below −10° C. Just prior to the experiment, the ground needle powder was ground once more in a standard household coffee mill (Type 4041, Model KSM2™ by Braun), sieved and dried at 60° C. for 4 hours in an air ventilation dryer with digital temperature control (Fisher Scientific, Model 737F™). The sieved needle powder was then mixed thoroughly to obtain homogenous needle powder.

All solvents used in the experiment were HPLC trade (EM Science, Gibbstown, N.J.). Silica gel particles between 32 and 63 μm, (Fisher Scientific, Selecto Scientific, Georgia, USA), were used, without any further treatment.

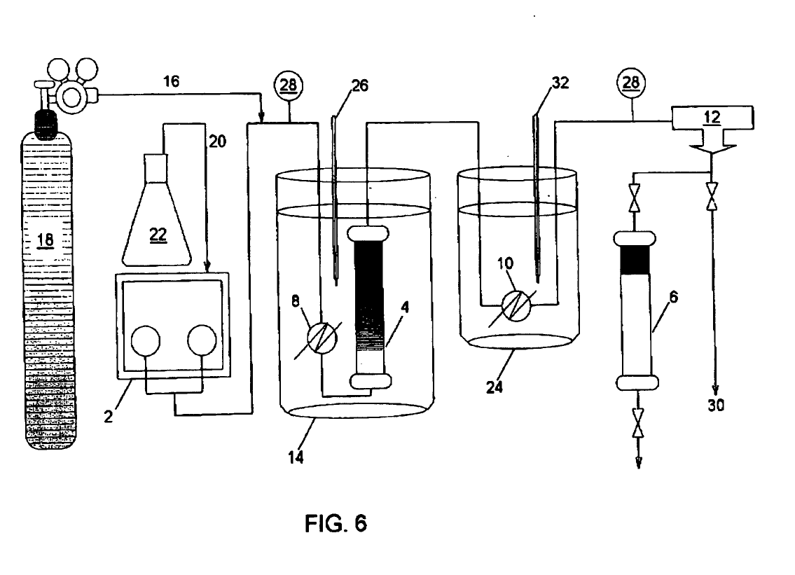

Dynamic Pressurized Liquid Extraction (DPLE) was carried out using the experimental setup shown in FIG. 6. A Waters™ 501 HPLC pump 2 was used, at a flow rate of between 0.0 ml/min to 9.9 ml/min. The extraction column 4 and solid phase extraction column 6 were Omnifit™ medium pressure preparative chromatography columns (15 mm inner diameter, 100 mm in length, pressure rate 300 psig) made of borosilicate glass with a fixed endpiece and an adjustable endpiece. The heat exchangers 8, 10 were 150 mm in Length, 2 mm in inner diameter and made of copper. The relief valve 12 was a Swagelock, Type RL₃™, with an adjustable realief pressure. The hot water bath 14 was an Ultra-Thermostat™, Model NB-35 703.

Hexane was selected as the extraction solvent to remove hydrophobic impurities from biomass. A 5.000 g sample of finer than 100-mesh needle powder was weighed and transferred into the extraction column 4. The height of the bed of the extraction column 4 was set to 4.5 cm by adjusting the adjustable endpiece of the column 4.

The hot water bath 14 was set at 90° C. and allowed to equilibrate before extraction experiment was conducted. The solvent 20 was purged with pure helium for 30 minutes using the HPLC online degassing system. After assembling the extraction system, the seals of the system were tested with pressurized nitrogen 16, from nitrogen tank 18. The system, including the extraction column 4, was purged with nitrogen 16. The relief valve 12 was adjusted to maintain a system pressure within 70-75 psig, to prevent the solvents from boiling.

The extraction column 4 and the heat exchanger 8 upstream of the extraction column 4 were immersed into the hot water bath 14 for 5 minutes so that the temperature of extraction column 4 reached the extraction temperature, as indicated by a hot bath thermometer 26. Solvent 20 from solvent reservoir 22 was then pumped at 1.0 ml/min through the system, which included the heat exchanger 8 in the hot water bath 14, the extraction column 4, the second heat exchanger 10 in a cold water bath 24 and the relief valve 12. Temperature of the cold water bath 24 was indicated by cold bath thermometer 32. Time recording started with the first drop of extract to appear out of the system. The extract and recovered solvent were collected at point 30 and the extract was analyzed for total weight and taxane content.

After 60 minutes of extraction, the system was purged with high pressure nitrogen (70-75 psig) in order to remove liquid solvent. Then the pressure of the system was reduced to ambient pressure and the system was purged with low pressure nitrogen (less than 10 psig) for 5 minutes to remove any solvent residue. Pressures were monitored by pressure gauges 28. At the final stage of extraction, the extraction column 4 and the upstream heat exchanger 8 were taken out of the hot water bath 14 and cooled in a fume hood (not shown). The residue of needle powder in the disassembled extraction column 4 was pushed out from the column using the adjustable endpiece, for further analysis.

The hexane extract from DPLE was kept in the fume hood at room temperature for 12 hours and a small amount of green precipitate was observed on the bottom of the test tube in which the hexane extract was collected. The precipitate was separated by filtration (not shown) and the filtrate was collected in a Petri dish and left in a fume hood for 12 hours. The paclitaxel content of both dried filtrate and precipitate were analyzed with HPLC.

There was found to be no paclitaxel detected in the dried filtrate (Table 1), indicating that that all of the paclitaxel extracted by DPLE with hexane was in the precipitate and readily separated from most lipids in the hexane extract.

TABLE 1
Hexane Extract Analysis after 12 hours
Precipitation at Room Temperature. DPLE Conditions:
1.0 ml/min, 90.0° C., 30 minutes.

| | Dried Filtrate | Precipitate |
|---|---|---|
| Net Weight (g) | 0.28345 | 0.02425 |
| Appearance/ | Dark brown, tar-like | Fine green powder, |
| Observations | semi-solid | readily dissolves in |
| | | dichloromethane or |
| | | methanol |
| Taxanes Content | No taxanes detected | Paclitaxel only, 80 μg |

### EXAMPLE II

The process of Example I was repeated, with the following exceptions:
1. The needle powder was extracted by DPLE with hexane for 30 min at 90° C. to remove lipids.
2. Dichloromethane was used as the solvent to extract taxanes from the pre-treated needle powder.
3. The resultant green precipitate was dissolved in dichloromethane extract.
4. The dichloromethane extract was left in the fume hood for 12 hours to remove solvent.

The solid in the dichloromethane extract was analyzed with HPLC. There was found to be 1789 μg paclitaxel, 3120 μg of 10-DAB III, 105 μg of Baccatin III and 3216 μg of 9-DHB III in the solid.

### EXAMPLE III

The process of Example II was repeated, with the following exceptions:
1. 5,000 g silica gel was weighed and packed in a Normal Phase Solid Phase Extraction (NP-SPE) column 6, as illustrated in FIG. 6.
2. Dichloromethane extract was fed into the NP-SPE column 6 instead of being collected with test tubes.
3. The eluate from the NP-SPE column 6 was collected in a Petri dish and dried in a fume hood for 12 hours. The solids in the Petri dish were dissolved in methanol for HPLC analysis after filtration through a 0.45 μm filter.

No taxanes were detected in the eluate from the NP-SPE column 6. In comparing this result to that of Example II, all of the taxane was collected within the NP-SPE column 6.

### EXAMPLE IV

The process of Example III was repeated, with the following exceptions:
1. The NP-SPE column 6 with taxanes was eluted with mixtures of from 70:30 to 20:80 dichloromethane:ethyl acetate.
2. Fractions were collected every 50 ml and analyzed for taxane contents with an HPLC. Those fractions containing taxanes were pooled together and kept in the fume hood for 12 hours. The resulting light-coloured solid was analyzed with HPLC for taxane content.

There were 1780 μg paclitaxel, 3131 μg 10-DAB III, 98 μg Baccatin III and 3135 μg 9-DHB III found in the solid.

### EXAMPLE V

The process of Example I was repeated, with the following exceptions:
1. Dichloromethane was used to extract lipids and taxanes in the needle powder.
2. The dichloromethane extract was mixed with 4.0 g Celite™ 545 and left in the fume hood for 12 hours to remove the solvent.
3. The mixture from Step 2 was transferred into a first Omnifit™ medium pressure preparative chromatography column (15 mm inner diameter, 100 mm in length, pressure rated to 300 psig, made of borosilicate glass with a fixed endpiece and an adjustable endpiece).
4. A second Omnifit™ medium pressure preparative chromatography column with the same dimension was packed with 5.0 g macropore resin (HP2MG™). The second column was elated with 50 ml of ethanol followed by 50 ml of water.
5. Mixtures of from 20:80 to 80:20 ethanol:water were forced to flow through the first column and then through the second column. The eluant fractions were collected and analyzed for taxanes content with an HPLC.

There were found to be 1713 μg of paclitaxel, 3009 μg of 10-DAB III, 95 μg of Baccatin III and 3125 μg of 9-DHB III in the ethanol/water mixture.

This detailed description of the method is used to illustrate the prime embodiment of the present invention. It will be obvious to those skilled in the art that various modifications can be made in the present method and that various alternative embodiments can be utilized. Therefore, it will be recognized that various modifications can be made in the method of the present invention and in the applications to which the methods are applied without departing from the scope of the invention, which is limited only by the appended claims.

1. A process of extracting taxanes from plant biomasis, comprising:
a) comminuting the biomass;
b) feeding the biomass into a pressurized liquid extraction unit;
c) contacting the biomass with a halogenated C₁ or C₂ alkane solvent at a temperature of 100° C. or less and at pressure sufficient to keep the solvent in liquid form, to produce a stream of taxanes and solvent;
d) cooling the stream of taxanes and solvent,
e) stripping the solvent from the taxanes; and
f) conducting liquid chromatography on the taxanes to purify the taxanes.

2. The process of claim 1, wherein the solvent is selected from the group consisting of methylene chloride and chloroform.

3. The process of claim 1, wherein the temperature employed for contacting the biomass with the solvent is from 50° C. to 100° C.

4. The process of claim 3, wherein the temperature employed for contacting the biomass with the solvent is from 80° C. to 100° C.

5. The process of claim 1, wherein the pressure for contacting the biomass with the solvent is less than 100 psig.

6. The process of claim 1, wherein the comminuted biomass is finer than 100 mesh.

7. The process of claim 1, wherein the liquid chromatography is normal phase liquid chromatography or reverse phase liquid chromatography.

8. The process of claim 1 which further comprises purging the dynamic pressurized liquid extraction unit pith an inert gas after dynamic pressurized liquid extraction, thereby removing any traces of the halogenated alkane solvent.

9. The process of claim 8 wherein the inert gas is nitrogen.

10. The process of claim 8 wherein the inert gas containing the halogenated alkane solvent is recovered by adsorption onto an active carbon fibre matrix.

11. The process of claim 1, which further comprises contacting the biomass with an organic solvent before dynamic pressurized liquid extraction, to extract lipids from the biomass.

12. The process of claim 11, wherein the organic solvent is selected from the group consisting of petroleum ether and hexane.

13. The process of claim 11, wherein the lipid is extracted at a temperature of from 70° C. to 100° C.

14. The process of claim 13, wherein the lipid is extracted at a temperature of from 90° C. to 100° C.

15. The process of claim 11 which further comprises purging the biomass with an inert gas before contacting the biomass with the organic solvent, thereby removing oxygen.

16. The process of claim 11 wherein the process further comprises purging the biomass with an inert gas after contacting the biomass with the organic solvent, to remove any traces of the organic solvent.

17. The process of claim 15 or 16 wherein the inert gas is nitrogen.

18. The process of claim 1, wherein said stripping of the solvent from the taxanes comprises:
a) feeding the stream of taxanes and solvent into a solid phase extraction column containing an adsorbent material;
b) adsorbing the taxanes onto the adsorbent material; and
c) collecting the solvent for reuse.

19. The process of claim 18 wherein the adsorbent material is selected from the group consisting of silica gel and Al₂O₂.

20. The process of claim 18 further comprising purifying the adsorbed taxanes by gradient elution.

21. The process of claim 20 wherein the adsorbed taxanes are eluted with methylene chloride.

22. The process of claim 18 further comprising purging the solid phase extraction column with an inert gas after step iii) to remove any traces of the solvent from the adsorbent material.

23. The process of claim 22 wherein the inert gas is nitrogen.

24. The process of claim 1, wherein said stripping of the solvent from the taxanes comprises:
a) feeding the stream of taxanes and solvent into a rotary dryer, together with a porous solid support material;
b) evaporating the solvent from the taxanes and condensing the solvent for reuse;
c) adsorbing the taxane products onto a surface of the support material;
d) loading the support material with the taxanes into a sample column;
e) eluting the sample column with an organic solvent/water mixture to remove taxanes from the support material;
f) conducting reverse phase chromatography on the taxanes and organic solvent/water mixture to adsorb the taxanes; and
g) conducting a gradient elution of the taxanes to obtain purified taxane products.

25. The process of claim 24 wherein the support material is diatomite.

26. The process of claim 24 wherein the reverse phase chromatography comprises passing the taxanes and organic solvent/water mixture through a reverse phase chromatography column packed with macro pore resins.

27. The process of claim 24 further comprising conducting membrane separation on the taxanes after reverse phase chromatography to further recover taxane products.

28. A process for extracting taxanes from plant biomass, comprising:
a) comminuting the biomass;
b) feeding the biomass into a pressurized liquid extraction unit;
c) contacting the biomass with a solvent select from the group consisting of methylene chloride and chloroform, at a temperature of 100° C. or less and at sufficient pressure to keep the solvent in liquid form, to extract a stream of taxanes and solvent;
d) cooling the stream of taxanes and solvent;
e) feeding the cooled stream into a solid phase extraction column containing an adsorbent material;
f) adsorbing the taxanes onto the adsorbent material;
g) collecting the solvent for reuse; and
h) conducting normal phase liquid chromatography on the taxanes.

29. A process for extracting taxanes from plant biomass, comprising:
a) comminuting the biomass;
b) feeding the biomass into a pressurized liquid extraction unit;
b) contacting the biomass with a solvent select from the group consisting of methylene chloride and chloroform, at a temperature of 100° C. or less and at sufficient pressure to keep the solvent in liquid form, to extract a stream of taxanes and solvent;
c) cooling the stream of taxanes and solvent;
d) feeding the cooled stream into a rotary dryer, together with a porous solid support material;
e) evaporating the solvent from the taxanes and condensing the solvent for reuse;
f) adsorbing the taxane products onto a surface of the support material;
g) feeding the support material with the taxanes into a sample column;
h) eluting the sample column with an organic solvent/water mixture to remove taxanes from the support material;
i) conducting reverse phase chromatography on the taxanes and organic solvent/water mixture to adsorb the taxanes;
j) conducting a gradient elution of the taxanes to obtain purified taxane products; and
k) conducting reverse phase liquid chromatography on the purified taxane products.

30. Use of dynamic pressurized liquid extraction in a process for extracting taxanes from plant biomass,

31. A process for selecting at least one solvent for extracting a product from plant biomass containing a plurality of compounds, comprising:
a) assessing relative hydrophobicity of the compounds;
b) arranging the compounds on a scale of most hydrophobic to least hydrophobic; and
c) matching the hydrophobicity of the product to hydrophobicity of the at least one solvent.
